(19) United States
(12) Patent Application Publication
(10) Pub. No.: US 2007/0270933 A1
(43) Pub. Date: Nov. 22, 2007

(54) STENT HAVING CONTOURED PROXIMAL END

(75) Inventors: Randolf Von Oepen, Los Altos, CA (US); Thomas Michael Rieth, Hirrlingen (DE); Lorcan James Coffey, Tubingen (DE); Richard Roy Newhauser, Redwood City, CA (US); Travis Richard Yribarren, Coarsgold, CA (US)

Correspondence Address: WORKMAN NYDEGGER, 1000 EAGLE GATE TOWER,, 60 EAST SOUTH TEMPLE, SALT LAKE CITY, UT, 84111, US

(73) Assignee: Abbott Laboratories, Santa Clara, CA (US)

(21) Appl. No.: 11/683,999

(22) Filed: Mar. 8, 2007

Related U.S. Application Data
(60) Provisional application No. 60/780,752, filed on Mar. 9, 2006.

(51) Int. Cl. A61F 2/06

(52) U.S. Cl. 623/1.11

(57) ABSTRACT

A contoured intraluminal stent for deployment proximate a bifurcated vessel is disclosed. The contoured stent is configured to seat within a side branch of the bifurcated vessel so as to acceptably cover a curvilinear ostium defined at the intersection of the side branch with a main branch of the vessel. A stent deployment system includes a first guidewire positioned in the vessel main branch and a second guidewire that extends through the main branch and into the vessel side branch, wherein the intersection of the branches defines an ostium having a three-dimensionally curvilinear shape. A balloon catheter is tracked along the second guidewire to a position in the side branch proximate the ostium. A contoured stent disposed about the balloon includes a proximal end that defines a curvilinear profile that at least approximately matches the curvilinear shape of the ostium when the stent is deployed in the side branch.

### CROSS-REFERENCE TO RELATED APPLICATIONS

This application claims the benefit of the U.S. Provisional Patent Application No. 60/780,752, filed Mar. 9, 2006, and entitled “Contoured Stent and Delivery System with Novel Tip Design,” which is incorporated herein by reference in its entirety.

### BACKGROUND

1. Technology Field

The present invention generally relates to stents and stent delivery systems, used in intravascular systems. In particular, the present invention relates to a stent that is shaped and configured for placement within a side branch portion of a bifurcated lumen so as to not interfere with an additional stent positioned in the main luminal branch.

2. The Related Technology

Angioplasty and stent implantation procedures are commonly employed to treat lesions or blockages that form within the vascular anatomy of a patient. During an angioplasty, or percutaneous transluminal coronary angioplasty (“PTCA”) procedure, for instance, a guiding catheter is advanced through the vasculature of the patient to a desired point, such as the ostium of a predetermined coronary artery. A guidewire, positioned within a balloon catheter, is extended from a distal end of the guiding catheter into the patient's coronary artery until it penetrates and crosses a lesion to be dilated. The balloon catheter is then advanced through the guiding catheter and over the previously introduced guidewire, until it is properly positioned across the lesion.

Once properly positioned, the balloon is inflated to a predetermined size such that the stenosis of the lesion is compressed against the arterial wall, thereby expanding the passageway of the artery. The balloon is subsequently deflated, blood flow resumes through the dilated artery, and the balloon catheter is removed.

Occasionally, post-procedure restenosis, or reformation of the arterial blockage, occurs after the PTCA procedure has been performed. To reduce the incidence of restenosis and strengthen the dilated area, physicians frequently implant an intravascular prosthesis, generally called a stent, inside the artery at the site of the lesion. During a stent implantation procedure, a stent is delivered in a contracted state on a balloon catheter to the desired location within a coronary artery. Once properly positioned, the stent is expanded to a larger diameter via expansion of the balloon, which causes the stent to expand against the arterial wall at the lesion site. The balloon is then deflated and it and the catheter are withdrawn. The expanded stent remains in place within the artery at the site of the dilated lesion, holding the vessel open and improving the flow of blood therethrough.

Lesions are often located at or near a point of bifurcation in an artery or other body vessel. When treating such bifurcated lesions, it is common to first place a first guidewire in the main branch, then place a second guidewire, extending from the main branch, into the side branch of the vessel bifurcation. This is so because it is generally important to preserve access to, and blood flow within, the side branch and the main branch of the bifurcation.

Specifically, in some instances the above-described dilation via PTCA procedure causes plaque to be shifted from the treated main branch of the vessel bifurcation to the non-treated vessel side branch, thereby occluding the side branch. This effect is known as the “snowplow” effect. Prior placement of the second guidewire in the vessel side branch enables treatment of the side branch should it become occluded due to the snowplow effect.

Treatment of the side branch in this case often includes deployment of a stent therein. The stent is desirably placed in the vessel side branch proximate the bifurcation and deployed so that its proximal end covers as much of the ostium, or vessel opening, as possible.

However, the ostium—which is defined by the intersection of the vessel side branch with the main branch at the point of bifurcation - does not typically define a simple circle, but rather a curvilinear, saddle-shaped profile. This curvilinear ostium profile is typically caused by the oblique angle of intersection of the substantially cylindrical main and side branch vessels. In contrast, known stents currently in use include proximal ends that define a simple flat circular profile.

The disparity between the stent proximal end and ostium profiles typically causes one of two unfortunate results. First, if it is desired for the known stent to cover the entirety of the vessel side branch ostium, the stent is only partially inserted into the lumen of the side branch, resulting in a portion of the proximal end extending from the lumen of the side branch into the lumen of the main branch. Such a placement is highly undesirable as the portion of the side branch stent extending into the main branch can interfere with the proper placement of a stent to be deployed in the main branch proximate or past the bifurcation region.

Alternatively, the stent may be positioned fully into the side branch so that no portion thereof remains in the lumen of the main vessel branch when deployed. However, this placement is also undesirable, for it leaves portions of the side branch ostium uncovered by the deployed stent and therefore susceptible to further degradation or formation of a stenosis.

As seen by the above discussion, therefore, it is sometimes necessary in the treatment of lesions at a bifurcated vessel site to deploy a stent in the side branch so that the stent structure covers the side branch vessel walls beginning from the ostium where the side branch departs from the main vessel branch to the end of the vessel intended for coverage by the stent structure.

Unfortunately, the design of known stents is incapable of acceptably covering the ostium region of bifurcated vessel side branches without causing the above-described complications, again, the failure of the proximal end of the stent to cover the entirety of the ostium region of the side branch, or—in order to cover the entirety of the side branch ostium—the stent proximal end overhangs into the lumen of the vessel main branch.

In light of the above discussion, a need exists for an intraluminal stent suitable for placement at bifurcated vessels. The stent should be advantageously configured so as to cover all portions of the ostium region of the vessel side branch. Furthermore, the stent should be designed so as to prevent stent overhang in the lumen of the vessel main branch so as not to impede the placement of another stent in the main branch.

### BRIEF SUMMARY

The present invention has been developed in response to the above and other needs in the art. Briefly summarized, embodiments of the present invention are directed to a contoured intraluminal stent for deployment proximate a bifurcated vessel. The contoured stent is configured to seat within a side branch of the bifurcated vessel so as to acceptably cover a curvilinear ostium defined at the intersection of the side branch with a main branch of the vessel.

A stent deployment system employed with the contoured stent includes a first guidewire positioned in the vessel main branch and a second guidewire that extends through the main branch and into the vessel side branch, wherein the intersection of the branches defines an ostium having a three-dimensionally curvilinear shape. A balloon catheter is tracked along the second guidewire to a position in the side branch proximate the ostium. A contoured stent disposed about the balloon includes a proximal end that defines a curvilinear profile that at least approximately matches the curvilinear shape of the ostium when the stent is deployed in the side branch.

Importantly, the contoured stent is deployed in the lumen of a vessel side branch such that subsequent placement of a second stent in the main branch proximate the vessel bifurcation is not inhibited by the contoured stent. This is so because the proximal end of the contoured stent is shaped so as to match the shape of the ostium of the side branch, thereby preventing overhang of the contoured stent proximal end into the lumen of the main branch. In other embodiments, the proximal and/or distal end of the stent can be shaped so as to match ostiums or vessel portions having other shape configurations.

These and other features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof that are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 is a perspective view of a bifurcated vessel region, showing the curvilinear ostium profile of a side branch with respect to the vessel main branch;

FIG. 2 is a perspective view of a stent having a contoured proximal end for use in a vessel side branch, according to one embodiment;

FIG. 3 is a perspective view of a profile defined by the proximal end of the contoured stent of FIG. 2;

FIG. 4 is a perspective view of the contoured stent of FIG. 2 after placement thereof in the vessel side branch of FIG. 1;

FIG. 5 is a perspective view of a system for installing the contoured stent, according to one embodiment; and

FIGS. 6A-6E depict various stages of use of the stent installation system of FIG. 5 in placing the contoured stent of FIG. 2 in a vessel side branch.

### DETAILED DESCRIPTION OF SELECTED EMBODIMENTS

Reference will now be made to figures wherein like structures will be provided with like reference designations. It is understood that the drawings are diagrammatic and schematic representations of exemplary embodiments of the invention, and are not limiting of the present invention nor are they necessarily drawn to scale.

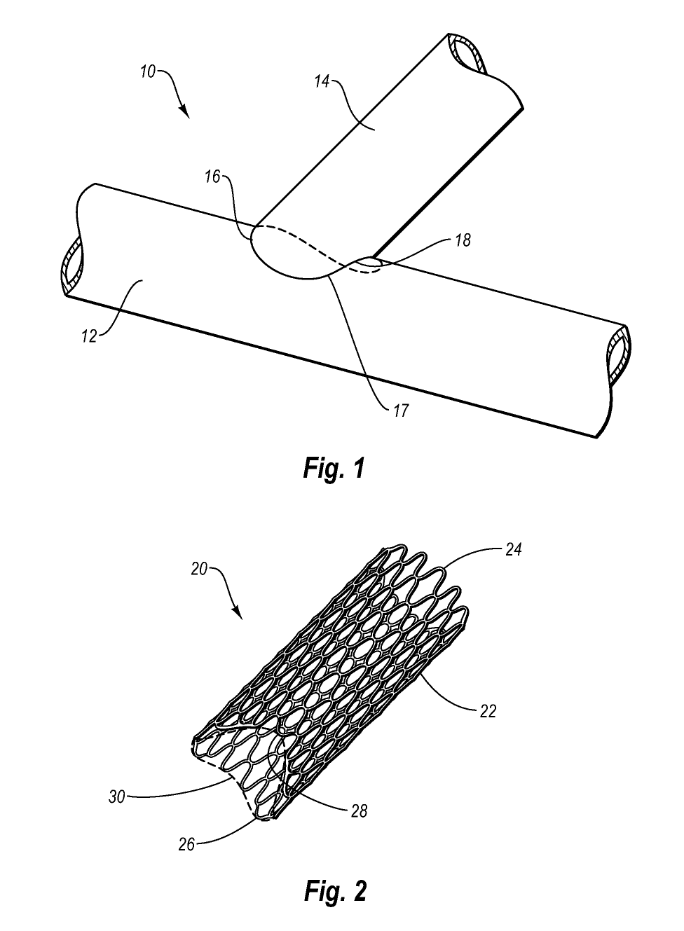

FIGS. 1-6E depict various features of embodiments of the present invention, which is generally directed to a contoured stent for use in intravascular treatment of lesions in a patient. The contoured stent includes a body defining a distal end and a proximal end. The proximal end of the stent defines a curvilinear profile that is configured to at least approximately match the curvilinear profile of an ostium of a vessel, such as a side vessel that extends from a main vessel at a point of vessel bifurcation. The matching profiles of the ostium and the stent proximal end enable the stent to be placed in the vessel side branch so as to substantially cover all portions of the ostium without a portion of the stent extending out into the main vessel lumen to obstruct other stents that may be placed at, near, or past the vessel bifurcation.

Reference is first made to FIG. 1 in describing an exemplary environment in which embodiments of the present invention can be practiced. In particular, FIG. 1 shows a vessel, generally designated at 10, including a main branch 12 and a side branch 14. The main branch 12 and side branch 14 define a bifurcation of the vessel 10, wherein the side branch extends from the main branch. An opening, or ostium 16 of the side branch 14 is defined at the point of bifurcation with the main branch 12.

Because of the oblique angle of intersection of the side branch 14 with the main branch 12, together with the generally cylindrical cross sectional shapes of the branches, the ostium 16 defines a three-dimensional curvilinear profile, approximating a saddle shape, as indicated by the line 17. Again, because of the nature of the intersection of the vessel side branch 14 with the main branch 12, the non-uniform curvilinear profile of the ostium 16 includes a carina, or inset, 18. The inset 18 presents a particular challenge for standard, known stents because of their inability to acceptably cover the inset and other regions of the ostium when deployed without causing other challenges.

In contrast, the stent disclosed in embodiments of the present invention is configured to deploy so as to acceptably cover all portions of the ostium 16. Particularly, FIG. 2 depicts a contoured stent, generally designated at 20, which is deployable for use with an ostium such as that shown at 16 in FIG. 1. As shown, the contoured stent 20 includes a generally cylindrical body 22 defining a distal end 24 and a proximal end 26. The body 22 of the stent is composed in the present embodiment of an interlocking lattice of small strand wire composed of a suitable material, such as stainless steel. The interlocking lattice of the stent body 22 is expandable for deployment within the lumen of the side vessel 14, as will be described. Notwithstanding its characterization herein, it is appreciated that the stent body can be configured in other ways from what is described herein while still residing within the scope of the claims.

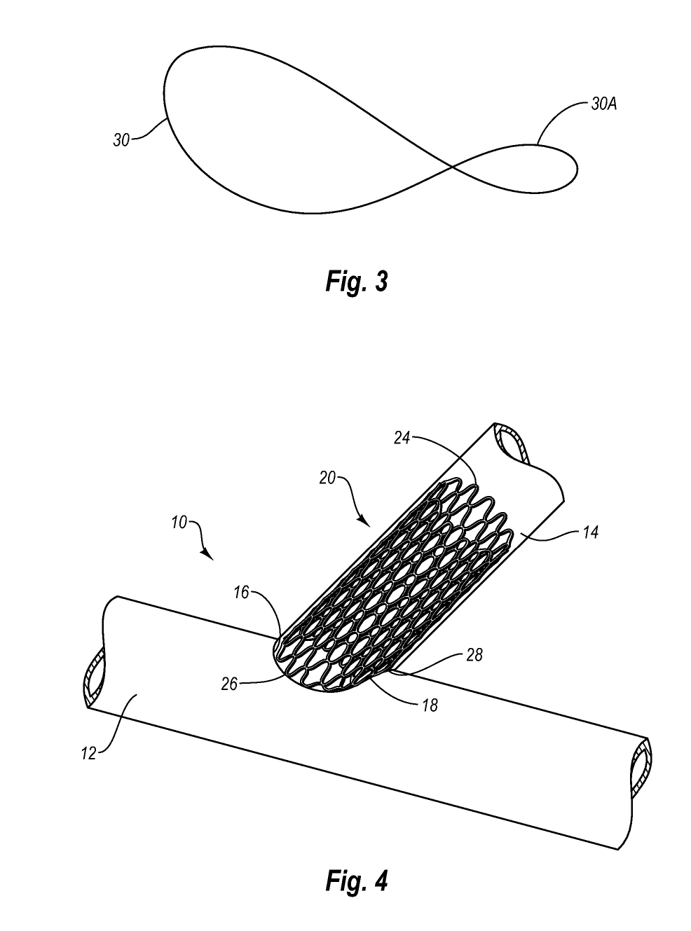

FIG. 2 shows that the proximal end 26 of the stent 20 includes an inset portion 28. The inset portion 28 generally defines a parabolic shape that extends toward the distal end 24 of the stent 20 a predetermined distance. As such, the inset portion 28 represents the most distal portion of the proximal end 26 of the stent 20. In greater detail, the outermost portions of the interlocking wire lattice of the stent body 22 body located at the proximal end 26 thereof generally define a three-dimensionally contoured profile 30. The profile 30 is more easily seen in FIG. 3, which shows the profile including an inset portion 30A that corresponds to the inset portion 28 defined by the stent proximal end. The contoured profile 30 is configured to at least approximately match the three-dimensionally curvilinear profile of the exemplary ostium 16 of the vessel side branch 14 shown in FIG. 1 so as to acceptably cover all portions of the ostium.

Note that the particular contour of the proximal end of the stent can be altered in shape and configuration from what is described herein so as to acceptably match ostiums of other vessels, both bifurcated and non-bifurcated, having other curvilinear shapes. For example, the profile of the stent proximal end in one embodiment can include two or more inset portions to acceptably match a similarly contoured vessel ostium when the stent is deployed in the lumen of the vessel. Or, the proximal end can define an elliptical shape, or other curvilinear or non-curvilinear shape. As such, the presently described embodiments should not be construed to limit the present invention in any manner.

Together with FIGS. 2 and 3, reference is now made to FIG. 4 in describing various details regarding the placement and deployment of the contoured stent 20. As shown, the contoured stent 20 is configured for placement in a lumen of the vessel side branch 14 of a bifurcated vessel in order to correct a stenosis or related condition in the vessel. Further, the contoured stent 20 is configured for positioning within the vessel side branch so as to completely cover the ostium 16 thereof. In this way, all portions of a stenosis that is formed at or near the bifurcation of the side branch 14 with the main branch 12 of the vessel 10 may be acceptably treated by the contoured stent 20.

As shown in FIG. 4, the contoured stent 20—shown deployed with its body 22 expanded against the vessel wall—is positioned such that the inset portion 18 thereof is disposed at a trailing portion of the intersection of the main branch 12 and side branch 14. So positioned, the deployed stent proximal end 26 follows along the profile of the ostium 16, delineated by the line 17, as desired. Further, the contoured stent 20 is placed such that it does not overhang into the lumen of the main vessel branch 12 such that subsequent placement of a second stent in the main branch is not impeded. In one embodiment, the contoured stent 20 has a length of approximately 15 mm, with a range of about 3 to 40 mm, at its longest point and a deployed diameter of approximately 2 mm, with a range of about 1 to 10 mm. The inset portion 28 of the contoured stent 20 extends from the proximal end 26 inward toward the distal end 24 a distance of approximately 2 mm, with a range of about 0.5 to 4 mm. Note, however, that these dimensions are exemplary only, and can change according to the patient, placement of the stent, or other factors.

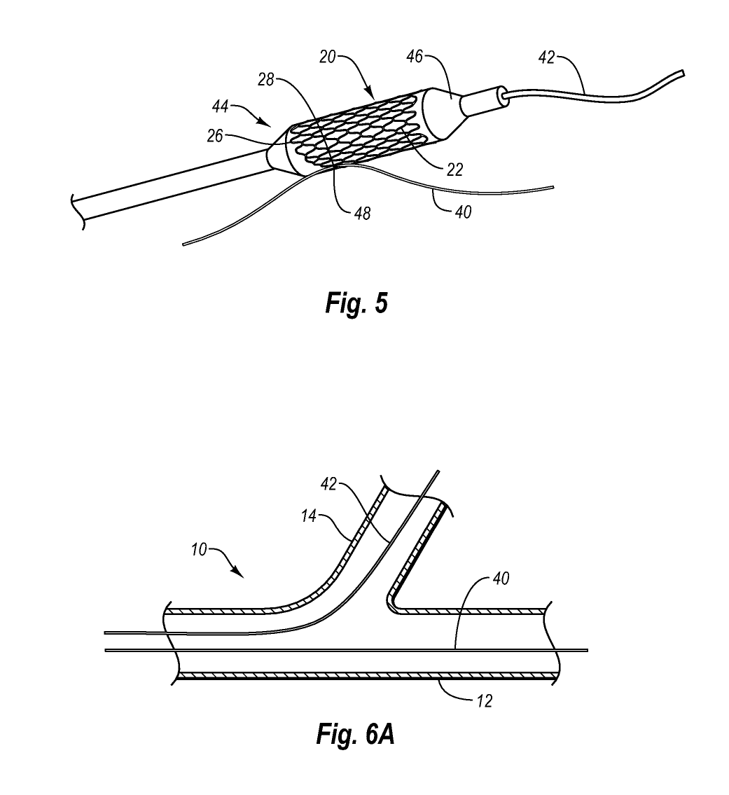

Reference is now made to FIG. 5 in describing details of a system for intraluminal placement and deployment of the contoured stent 20, according to one embodiment. The deployment system shown in FIG. 5, shown disposed in the lumen of the vessel main branch 12 before arrival and placement of the stent at the bifurcation location shown in FIG. 4, includes a first guidewire 40 and a second guidewire 42 disposed in the main branch lumen. A balloon catheter 44 including a balloon 46 tracks along the second guidewire 42 in the lumen.

The contoured stent 20 is included about the balloon 46 in its collapsed state, prior to deployment at the lesion site. The stent proximal end 26 is disposed proximally in the lumen of the vessel main branch 12. As shown, the first guidewire 40 passes through the stent body 22 at a location 48 that is proximate a distal edge, or tip, of the inset portion 28 of the stent. Positioned in this manner, the first guidewire 40 is capable of orienting the contoured stent 20 into a desired orientation within the vessel lumen as the balloon catheter 44 travels over the second guidewire 42 via the vessel lumen(s) to the bifurcation location where the stent will be ultimately positioned in the vessel side branch 14.

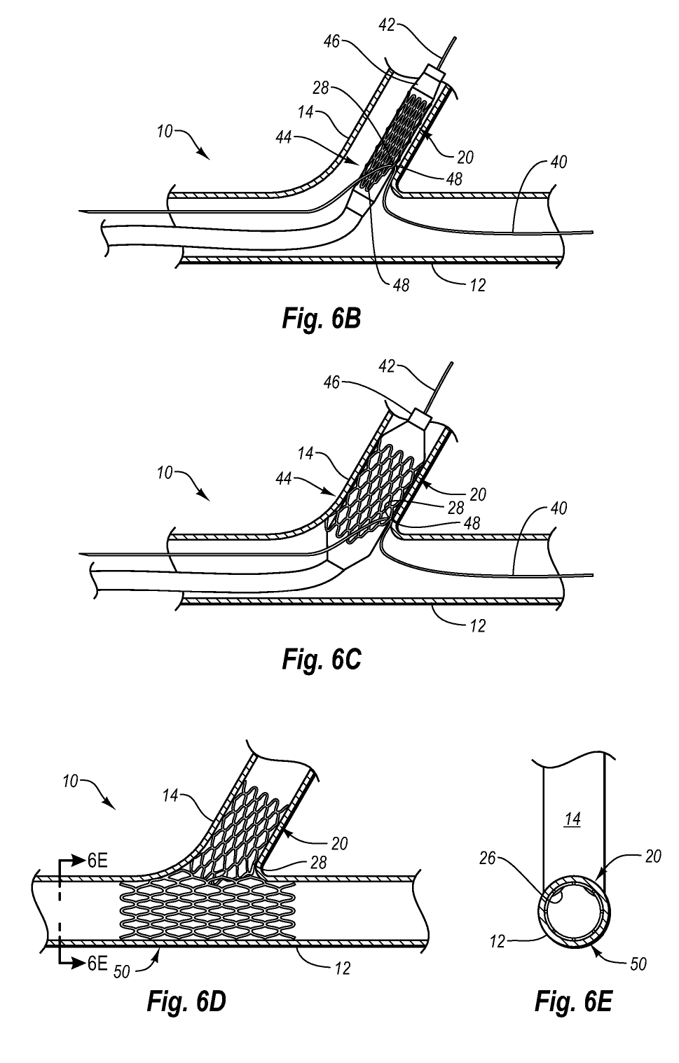

Specifically, orientation of the contoured stent 20 by the first guidewire 40 is accomplished in one embodiment by maintaining a desired amount of tension between the first guidewire and the contoured stent 20 disposed about the balloon 46 of the balloon catheter 44 as the balloon catheter is tracked along the second guidewire 42. This tension, together with proper positioning of the first guidewire 40 in the vessel lumen, exerts a force on the contoured stent 20 such that the balloon catheter and stent are rotated as needed about a longitudinal axis of the balloon catheter so as to maintain the inset portion and rest of the contoured stent 20 in a desired orientation. The first guidewire 40 can exert a continual force on the contoured stent 20 so as to maintain the desired stent orientation during intraluminal advancement of the balloon catheter 44. Maintaining the desired contoured stent orientation is beneficial for properly placing the stent at the vessel bifurcation, as will be seen in connection with the discussion of FIGS. 6A-6D. Notwithstanding the present discussion, it is appreciated that other possible delivery modes for the contoured stent are also contemplated, and as such, no intention is made here to limit the present invention to only the above-described stent deployment system.

Reference is now made to FIGS. 6A-6D in describing various details regarding placement and deployment of the contoured stent 20 at a point of vessel bifurcation using the deployment system described above in connection with FIG. 5, according to one embodiment. FIG. 6A shows the first and second guidewires 40 and 42 positioned in the vessel 10. In particular, the first guidewire 40 extends within the lumen of the main branch 12 of the vessel, while the second guidewire 42 extends from the main branch into the side branch 14 of the vessel 10, in preparation for advancement of the balloon catheter 44.

In FIG. 6B the balloon catheter 44, having the contoured stent 20 crimped thereon, is tracked along the second guidewire 42 until positioned proximate the point of bifurcation of the vessel side branch 14 with the main branch 12. During tracking of the balloon catheter 44, the first guidewire 40 is used, as described in connection with FIG. 5, to place and/or maintain the contoured stent 20 in a desired orientation with respect to its final placement position proximate the ostium 16 of the vessel side branch 14.

In detail, it is desired that the contoured stent 20 be positioned such that its inset portion 28 is aligned with the inset 18 of the side branch ostium 16. In this way, the proximal end 26 of the contoured stent 20 is oriented as desired in order to cover the entirety of the ostium region of the vessel side branch 14 while not extending into the lumen of the main branch 12. Again, the first guidewire 40, received through the portion 48 of the stent body 22, is used to ensure such desired positioning of the stent 20 while still crimped on the balloon catheter 44.

FIG. 6C shows deployment of the contoured stent 20 by inflation of the balloon 46 of the balloon catheter 44 once the stent has been properly positioned in the vessel side branch 14, as described above. Inflation of the balloon 46 causes expansion of the stent body 22 such that it seats against the wall of the vessel side branch 14. Expanded in this manner, the proximal end 26 of the contoured stent 20 is positioned so as to substantially cover the ostium 16 of the vessel side branch 14 and the adjacent portion of the side branch extending distally therefrom.

In FIG. 6D, a second, standard stent 50 is shown deployed in the vessel main branch 12 at the vessel bifurcation, proximate the contoured stent 20 disposed in the side branch 14. Because of its curvilinearly contoured proximal end 26, the stent 20 does not inhibit the placement or deployment of the second stent 50, in contrast with known stents similarly placed in the vessel side branch 14. The perceived overhang shown in FIG. 6D is actually an outer lying portion of the stent that is seated against the side vessel wall, thereby avoiding interference with the second stent 50. The positional relationship of the two stents 20 and 50 is also seen in FIG. 6E. Note that placement of the stent 50 with the contoured stent 20 in the manner shown here enables all portions of the vessel bifurcation to be acceptably stented, thereby improving the effect of the stenting procedure.

Note that configuration of the contoured stent or associated deployment system can be modified from what is described herein, while still residing within the claims of the present invention. For instance, the contoured stent can be configured such that the first guidewire passes through another portion of the stent other than at the distal apex of the inset portion 28. These and other modifications are therefore contemplated as part of the present invention.

Details regarding yet another system for deploying the contoured stent of the present invention can be found in the U.S patent application entitled “SYSTEM AND METHOD FOR DELIVERING A STENT TO A BIFURCATED VESSEL,” filed Mar. 8, 2007, (attorney docket no. 17066.33.3), which is incorporated herein by reference in its entirety.

The present invention may be embodied in other specific forms without departing from its spirit or essential characteristics. The described embodiments are to be considered in all respects only as illustrative, not restrictive. The scope of the invention is, therefore, indicated by the appended claims rather than by the foregoing description. All changes that come within the meaning and range of equivalency of the claims are to be embraced within their scope.

What is claimed is:

1. An intraluminal stent, comprising:
a body defining a distal end and a proximal end, at least one of the ends of the body defining a profile having at least one three-dimensionally curvilinear portion.

2. The intraluminal stent as defined in claim 1, wherein the at least one curvilinear portion is included on the proximal end of the body and at least approximately matches a shape defined by an ostium of a vessel lumen in which the stent is to be deployed.

3. The intraluminal stent as defined in claim 2, wherein the ostium shape is defined by an intersection of the vessel in which the stent is to be deployed with another vessel.

4. The intraluminal stent as defined in claim 2, wherein the at least one curvilinear portion includes an inset portion that extends toward the distal end of the body.

5. The intraluminal stent as defined in claim 4, wherein the inset portion defines a parabolic shape.

6. The intraluminal stent as defined in claim 5, wherein a portion of the body receives a first guidewire therethrough, the first guidewire operable to orient the stent in a desired orientation in the vessel lumen in which the stent is to be deployed.

7. The intraluminal stent as defined in claim 6, wherein the portion of the body that receives the first guidewire is proximate the inset portion.

8. An intraluminal stent deployment system, comprising:
a first guidewire for placement in a first lumen of a first vessel;
a second guidewire for extension through the first lumen of the first vessel into a second lumen of a second vessel, the intersection of the second lumen with the first lumen defining an ostium having a three-dimensionally curvilinear shape;
a balloon catheter including a balloon for tracking along the second guidewire to a position in the second lumen proximate the ostium; and
a contoured stent disposed about the balloon of the balloon catheter, the stent including a proximal end that defines a curvilinear profile that at least approximately matches the curvilinear shape of the ostium when the stent is deployed in the second vessel lumen.

9. The stent deployment system as defined in claim 8, wherein the curvilinear profile of the stent proximal end includes an inset portion that extends toward a distal end of the stent.

10. The stent deployment system as defined in claim 9, wherein the first guidewire passes through a portion of the stent proximate the inset portion.

11. The stent deployment system as defined in claim 10, wherein the first guidewire is employed to orient the stent in the first lumen.

12. The stent deployment system as defined in claim 9, wherein the inset portion at least approximately defines a parabolic shape.

13. The stent deployment system as defined in claim 8, wherein the stent includes a body defined by a wire lattice, the wire lattice defining the curvilinear profile of the proximal end.

14. The stent deployment system as defined in claim 8, wherein the three-dimensional curvilinear shape of the ostium is a saddle, and wherein the curvilinear profile of the stent proximal end is a saddle.

15. The stent deployment system as defined in claim 8, wherein the first vessel is a main branch, and wherein the second vessel is a side branch that extends from the main branch at a point of bifurcation, the ostium being defined at the point of bifurcation.

16. The stent deployment system as defined in claim 15, wherein the stent is positioned so as to cover the ostium of the side branch without interfering with a second stent positioned proximate the point of bifurcation in the main branch.

17. A method for delivering a stent to a lumen of a second vessel extending from a bifurcation with a first vessel, the method comprising:
placing a first guidewire in a lumen of the first vessel;
placing the second guidewire in the lumen of the first vessel such that it extends into the second lumen;
passing the first guidewire through a portion of a stent;
tracking a catheter including the stent along the second guidewire toward a deployment position in the lumen of the second vessel proximate the ostium;
by the first guidewire passing through the portion of the stent, orienting the stent included on the catheter; and
deploying the stent in the deployment position.

18. The method for delivering as defined in claim 17, wherein deploying the stent further comprises:
deploying the stent in the deployment position such that a proximal end of the stent is positioned proximate an ostium of the second vessel and such that the stent does not inhibit placement of a second stent in the first vessel proximate the bifurcation.

19. The method for delivering as defined in claim 18, wherein the ostium defines a non-uniform shape, and wherein deploying the stent further comprises:
deploying the stent in the deployment position such that a proximal end of the stent that at least approximately defines the non-uniform shape of the ostium is positioned proximate the ostium.

20. The method for delivering as defined in claim 18, wherein the ostium defines a saddle shape, and wherein the stent when deployed in the second vessel includes a proximal end at least approximately defining a saddle shape.

21. The method for delivering as defined in claim 18, wherein passing the first guidewire through a portion of the stent further comprises:
passing the first guidewire through a portion of the stent proximate an inset portion defined by the proximal end of the stent.
